(19) United States
(12) Patent Application Publication
(10) Pub. No.: US 2007/0167517 A1
(43) Pub. Date: Jul. 19, 2007

(54) Stabilized derivatives of ascorbic aicd

(75) Inventors: Emma Kvitnitsky, Kiryat Shmona (IL); Valery Belakhov, Haifa (IL); Vladimir Babtsov, Kiryat Shmona (IL); Vury Shapiro, Givat Shmuel (IL)

Correspondence Address: BROWDY AND NEIMARK, P.L.L.C.;624 NINTH STREET, NW, SUITE 300, WASHINGTON, DC, 20001-5303, US

(73) Assignee: Tagra Biotechnologies Ltd., Netanya (IL)

(21) Appl. No.: 10/553,757

(22) PCT Filed: Apr. 21, 2004
(86) PCT No.: PCT/IL04/00343

§ 371 Date: Jan. 3, 2007

(30) Foreign Application Priority Data
Apr. 21, 2003 (US) 60464097

(51) Int. Cl. A61K 31/34; A01N 43/08

(52) U.S. Cl. 514/474

(57) ABSTRACT

Ascorbic acid derivatives of the general formula 1: wherein R<1> is a C2-C22 acyl group, an amino acid group, or a C1-C17 alkyl group; R<2> is ammonium or a metal cation; and each of R<3> or R<4>, independently, is hydrogen, a C2-C22 acyl group, an amino acid residue, or a C1-C17 alkyl group, are more stable than ascorbic acid and can be used as a source of vitamin C in pharmaceutical, nutraceutical and cosmetic compositions.

### FIELD OF THE INVENTION

The present invention relates to novel stabilized ascorbic acid derivatives and to compositions comprising them.

### BACKGROUND OF THE INVENTION

Vitamin C (ascorbic acid) is a vital nutrient for humans and has many important functions in-the body. Vitamin C is essential for collagen synthesis and helps maintain the integrity of substances of mesenchymal origin, such as connective tissue, osteoid tissue, and dentin. An essential function of ascorbic acid is to act as a cofactor for the hydroxylation of proline and lysine residues in collagen, a major protein component of the body, that is important in maintaining healthy skin elasticity and texture. Ascorbic acid is further essential for wound healing and facilitates recovery from burns. Being a strong reducing agent, ascorbic acid is reversibly oxidized and reduced in the body, functioning as a redox system in the cell and being useful in the treatment of cancer. It is involved in the metabolism of phenylalanine and tyrosine. Vitamin C facilitates the absorption of iron and protects folic acid reductase, which converts folic acid to folinic acid, and may help release free folic acid from its conjugates in food.

Vitamin C is a powerful antioxidant, protecting against oxidative damage to DNA, membrane lipids and proteins. As mentioned above, it is involved in the synthesis of numerous substances such as collagen, and also of certain anabolic steroid hormones, and transmitters of the nervous system, lipids and proteins. It seems to be required for proper immune function and its use has been recommended to prevent or treat colds. Although this has not been demonstrated by experimental studies, it seems that Vitamin C does shorten or reduce the severity of a cold.

Vitamin C is also a water-soluble cellular antioxidant that reacts with free radicals in the water compartment of cells and in intercellular fluids and can “recycle” vitamin E by chemically regenerating it after it has been spent in terminating a free radical reaction.

Vitamin C has been used in recent years as an active ingredient of cosmetics. Due to its antioxidant properties, it is considered to confer both antioxidant and photoprotection to skin against free radical attack and UV ray damage. Since free radicals are considered to be responsible for skin damage and premature ageing, cosmetics containing Vitamin C or derivatives thereof are currently marketed as anti-ageing agents to prevent skin free-radical damage by UVA rays, and to renew skin elasticity and firmness, through production of collagen, the body's intercellular cement, and other supporting structures.

Vitamin C deficiency causes scurvy that is characterized by capillary fragility resulting in bruising and hemorrhaging, inflammation of the gums, loosening of the is teeth, anemia and general debility that can lead to death. In some conditions, it may be recommended to administer increased vitamin C amounts, for example for people taking estrogens, oral contraceptives, barbiturates, tetracyclines, aspirin, and for cigarette smokers.

Many fruits and vegetables contain vitamin C. However to ensure an adequate amount in the daily diet, it is recommended that supplemental vitamin C be provided.

The formulation of pure vitamin C into an end product, however, presents serious difficulties because it is easily oxidized. Upon storage or exposure to light, oxygen, moisture and/or high temperature, vitamin C undergoes rapid degradation. It is unstable in aqueous solution, even under neutral pH and at room temperature. It also reacts with certain minerals, such as iron and copper salts, which contribute to a more rapid degradation.

In order to overcome the problem of the lack of stability of ascorbic acid in its pure form, derivatives thereof have been proposed that are more stable. According to one approach, stabilization of ascorbic acid and its hydrophobic derivatives is achieved after entrapment into cyclodextrins, zeolites or liposomes.

Another approach consists of stabilizing ascorbic acid by derivatization of its enediol function. Ascorbic acid in its lactone form has four hydroxyl groups at carbons 2, 3, 5 and 6. These hydroxyl groups have different chemical activities: the 2- and 3-hydroxyl groups, together with the double bond connecting carbons 2 and 3, form an enediol system that is very sensitive to oxidation and is responsible for the oxidative degradation of ascorbic acid, whereas the 5- and 6-hydroxyl groups form a rather stable-diol system. Common derivatization of ascorbic acid converts the hydroxyl groups to alkyl-, acyl-, sulfo-, or phosphoryl-containing groups, which also affect the solubility of ascorbic acid in water or in oils. Known ascorbic acid derivatives fall into two main groups, water-soluble and oil-soluble ascorbic acid derivatives. These two groups differ in their potential uses.

Stabilization of ascorbic acid by derivatization of its enediol function at the 2-position has been attempted by preparation of L-ascorbic acid 2-phosphate or L-ascorbic acid 2-sulfate. The ascorbic acid. 2-phosphate derivative did show some biological activity, but the ascorbic acid 2-sulfate was not found to be an effective biological agent.

In recent years, significant steps have been made to introduce vitamins and other active agents into dermatological or cosmetic compositions. Vitamin C has been particularly contemplated for dermatological and cosmetic purposes. By stimulating and regulating the synthesis of collagen, vitamin C increases the elasticity and structural integrity of human skin and inhibits the formation of wrinkles. Another important benefit of ascorbic acid is its protective effect against oxidative damage to the skin. A major cause of cutaneous damage is the generation of reactive oxygen species by chemical pollutants, smoking, and particularly ultraviolet (UV) radiation. Reactive oxygen species can damage lipids, proteins, and nucleic acids in skin cells, which in turn can lead to the development of cutaneous cancer and photoaging. Numerous investigators have reported that high levels of ascorbic acid in the skin, produced by topical application of the vitamin, provides some degree of protection against UV radiation, particularly UVA (Darr, D., S Dunston, H. Faust, and S. Pinnell. 1996. Acta Derm. Venereol. (Stockh). 76: 264-268; Black. H. S., and J. T. Chan. 1975. J. Invest. Dermatol. 65: 412-414). This protective effect is apparently due to the scavenging of oxygen free radicals generated in the cytosol by UVA rays.

Thus, topical compositions comprising vitamin C may be used for specific treatments of various skin problems such as aging, dryness, and acne or pigmentation disorders. The beneficial effects of ascorbic acid for topical application, particularly those resulting from its antioxidant activity, are optimized only if high levels of vitamin C are established and maintained in the tissues (Uacques, P. F., and L. T. Chylack. 1991. Am. J. Clin. Nutr. 53: 352S-355S). Increased ingestion of vitamin C has not resulted in increased skin levels of ascorbic acid because vitamin C concentration of the skin is regulated, and limited, by active transport mechanisms (Rumsey, S. C., and M. Levine. 1998. J. Nutr. Biochem. 9: 116-130). Although the direct delivery of a high concentration of ascorbic acid through the stratum corneum barrier into the skin may effectively increase the pool of protective antioxidants and enhance collagen synthesis, there are many obstacles that must be overcome to reach this goal. For example, as a water-soluble molecule, vitamin C is not stored well in the tissues and is rapidly removed from the body, with a half-life of about 20 days in the human (Ritchey, S. J. 1965. Am. J. Clin. Nutr. 17: 57-114). Also, because of its hydrophilic nature, vitamin C is not absorbed well into specific tissues, especially the skill, which naturally repels water and water-soluble substances. Depending on the vehicle used, only about 8% of topically-applied ascorbic acid is actually absorbed into the skin (Darr, D., S. Combs, S. Dunston, T. Manning, and S. Pinnell. 1991. J. Invest. Dermatol. 96: 590). Naturally, vitamin C incorporated into various compositions has to be strongly protected against oxidation. Vitamin C derivatives are available in forms suitable for any skin type, but it is critically important to use a stabilized form that can actually penetrate the skin to have its protective anti-oxidant effect.

Among the stabilized derivatives for use in topical formulations, one can find the 6-ascorbyl palmitate, a highly bioavailable, fat-soluble derivative of ascorbic acid that possesses all the benefits of vitamin C, but, unlike the water-soluble form, can be stored in the lipid cell membrane until its use by the body. Another well-known vitamin C derivative is the magnesium L-ascorbic acid-2-phosphate (MAP), a water-soluble, stable, non-poisonous and non-irritating derivative used as an additive in the modem functional whitening cosmetics, and said to remove spots by inhibition of tyrosinase activity, to remove wrinkles by elimination of free oxygen radical after the absorption by the skin, and to have a synergic effect with vitamin E.

The stability of ascorbic acid, ascorbyl palmitate and magnesium ascorbyl phosphate in both standard solutions and topical formulations was investigated by direct RP-HPLC analysis after sample dilution with a suitable aqueous-organic solvent mixture. The results showed that, whereas the two vitamin C derivatives were more stable than ascorbic acid, the ascorbyl esters showed significant differences. Esterification with palmitic acid in position 6 did not prevent hydrolysis of the molecule, either in solution or in emulsion; only the special preparation of products with high viscoelastic properties was able to reduce the typical behavior of this compound. Conversely, the introduction of the phosphoric group in position 2 protected the molecule from break-up of the enediol system, confirming that magnesium ascorbyl phosphate is a very stable derivative of vitamin C that may be easily used in various types of cosmetic products (Austria, R., Semenzato, A., Bettero, A. Stability of vitamin C derivatives in solution and topical formulations. J. Pharm. Biomed Anal. 1997, 15(6), 795-801).

U.S. Pat. No. 5,098,898 describes the coupling of glycerol ester or ether to L-ascorbic acid via a phosphoric acid residue. The resulting compounds exhibits good antioxidant activity as well as lipid peroxide inhibiting activity. However, the enediol system is not adequately protected and thus cannot be used as a source of vitamin C in topical formulations.

JP 63104971 and DE 3613590 disclose synthesis of 2,3-di-O-acyl L-ascorbic acids, which are more photostable than the above-mentioned phosphorylated L-ascorbic acids. However, 2,3-di-O-acylation leads to loss of biological activity and bioavailability due to the low solubility of the products in water. Therefore, such compounds are practically useless for cosmetic, dermatological and other applications.

JP 7017989, JP 8034791, JP 98363316 and JP 98201242 disclose applications of L-ascorbyl-2-phosphate in chemical peeling and skin-whitening compositions to prevent penetration of the agents to skin in depth and reduce skin irritation. 2-Phosphate containing derivatives of L-ascorbic acid show appropriate stability and preserve own activity.

U.S. Pat. No. 3,671,549 and DE 1805958 disclose synthesis of L-ascorbyl-3-phosphate by direct phosphorylation of ascorbic acid with a phosphorus halide, phosphoric acid or halophosphoric acid or their corresponding anhydrides. The process is suitable for large-scale production.

JP 10324627 discloses synthesis of L-ascorbic acid derivatives having phosphate, pyrophosphate, triphosphate, polyphosphate, sulfate, or glycosyl groups in position 2, and hydroxyl, phosphate, polyphosphate, sulfate, glycosyl, alkoxyl, alkenyloxyl or phenoxyl groups in position 3, for their application as antitumor drugs. The problem of stability was not an aim of this patent but the new pharmacological activity only. Therefore, position 2 was not O-substituted with a residue effective enough for the oxidative protection of the enediol double bond.

U.S. Pat. No. 5,516,919 describes a method for the production of metal salts or optionally substituted ammonium salts of ascorbic acid derivatives by treating an acidic aqueous solution containing ascorbic acid 2-phosphate or ascorbic acid 2-sulfate with a porous adsorbent such as activated carbon, followed-by treating the adsorbent with a basic aqueous solution containing e.g. a metal salt of an organic acid or substituted or non-substituted ammonium salt ion, to obtain the end product of high quality in high yield by simple and easy operations.

U.S. Pat. No. 6,110,477 discloses a topical composition for preventing and/or treating photo-aged skin, sunburn, wrinkles and related skin disorders comprising ascorbic acid or a derivative thereof in a substantially anhydrous single phase carrier pharmaceutically acceptable base. The ascorbic acid derivatives disclosed are ascorbic palmitate, sodium ascorbate, potassium ascorbate, ammonium ascorbate, triethanolamine ascorbate, ascorbyl phosphate or magnesium ascorbyl phosphate.

U.S. Pat. No. 6,602,906 discloses the use of 5,6-O-isopropylidene-L-ascorbic acid for topical treatment of the skin for increasing the concentration of ascorbic acid in the dermal layer of the skin, for enhancing the synthesis of skin collagen, and for increasing the antioxidant potential of the skin.

### SUMMARY OF THE INVENTION

It is an object of the present invention to provide stabilized ascorbic acid derivatives which are substantially more stable over extended periods of storage than ascorbic acid itself.

The present invention relates to compounds of the general formula (I): 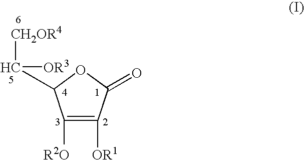
wherein R¹ is a C₂-C₂₂ acyl group, an amino acid residue, or a C₁-C₁₇ alkyl group; R² is ammonium or a metal cation; and each of R³ or R⁴, independently, is hydrogen, a C₂-C₂₂ acyl group, an amino acid residue, or a C₁-C₁₇ alkyl group.

The invention further provides a method for the preparation of the compounds of the formula (I).

The invention still further provides compositions comprising a compound of the formula (I).

### DETAILED DESCRIPTION OF THE INVENTION

The compounds of formula (I) of the present invention are ascorbic acid derivatives that are more stable as compared to ascorbic acid. The stability of these derivatives stems from the fact that simultaneous derivatization of the 2- and 3-hydroxyl groups protects the 2,3-enediol system.

The ascorbic acid derivatives of formula I serve as reliable, versatile and effective source of ascorbic acid to human tissues. Following their hydrolysis in situ, for example by enzymes present in tissue, e.g. skin, they can serve as a source of carboxylic acids or amino acids.

In accordance with the invention, R² is ammonium or a metal cation that may be chosen from any suitable, monovalent, divalent or trivalent metal cation.

In one preferred embodiment of the invention, R² is ammonium or a monovalent metal cation such as Na⁺, K⁺, or Li⁺. In another embodiment of the invention, R² is a divalent alkaline earth metal cation such as Mg⁺⁺, Ca⁺⁺, or Ba⁺⁺, or a trivalent metal cation such as Al⁺⁺⁺ or Fe⁺⁺⁺. In preferred embodiments, R² is selected from Na⁺, K⁺, Mg⁺⁺, or Ca⁺⁺.

As defined herein, the term “C₂-C₂₂ acyl group” refers to a carboxylic acyl group derived from a straight or branched, saturated or unsaturated carboxylic acid such as, but not limited to, acetic, propionic, n-butyric, isobutyric, sorbic, n-valeric, isovaleric, caproic, caprylic, capric, undecanoic, lauric, tridecanoic, myristic, pentadecanoic, palmitic, palmitoleic, stearic, oleic, linoleic, linolenic, nonadecanoic and arachidonic acid. In one preferred embodiment, the acyl group is C₈-C₁₈ acyl, more preferably capryloyl or is palmitoyl.

As defined herein, the term “C₁-C₁₇ alkyl group” refers to a straight or branched alkyl: group such as, but not limited to, methyl, ethyl, n-propyl, isopropyl, n-butyl, isobutyl, sec-butyl, t-butyl, n-pentyl, 1-methylbutyl, 2-methylbutyl, 3-methylbutyl, 2,2-dimethylpropyl, 1-ethylpropyl, n-hexyl, n-heptyl, n-octyl, n-nonyl, n-decyl, n-undecyl, n-dodecyl, n-tridecyl, n-tetradecyl, n-pentadecyl, n-hexadecyl and n-heptadecyl.

As defined herein, the term “amino acid” refers to a natural or non-natural amino acid, preferably a natural ,-amino acid such as glycine, alanine, serine, leucine, isoleucine, phenylalanine, tyrosine, glutamic acid, and the like. In one preferred embodiment, the amino acid is glycine.

In order for the compounds to serve as the biologically accessible source of ascorbic acid, R¹, R³ and R⁴ are preferably each independently chosen from natural and physiologically compatible moieties such as residues from saturated and unsaturated fatty acids or from amino acids.

For some uses, in order to achieve a relatively high percentage of liberated ascorbic acid after hydrolysis in the tissue, the overall molecular weight of the compounds of formula I should be kept rather low. Thus, in one preferred embodiment, R³ and R⁴ are both hydrogen since, preferably, although not essentially, no derivatization is needed for the 5- and 6-hydroxyls as these groups are rather stable and derivatization may unnecessarily increase the molecular weight of the ascorbic acid derivative. Such an increase in molecular weight may decrease the amount of liberated ascorbic acid per unit weight of compound. At times, however, such increase in molecular weight may be beneficial.

Thus, in one preferred embodiment of the invention, R³ and R⁴ are both hydrogen, R² is ammonium, a monovalent metal cation such as Na⁺ or K⁺, or a divalent alkaline earth metal cation such as Mg⁺⁺ or Ca⁺⁺, and R¹ is an acyl group derived from a C₆-C₂₂, preferably C₈-C₁₈, saturated or unsaturated fatty acid. In one preferred embodiment, R¹ is capryloyl. In another preferred embodiment, R¹ is palmitoyl.

Enzymes in tissues such as those of skin cells can easily hydrolyze such ascorbic acid derivatives of the invention and all the hydrolysis products are natural compounds and may thus have a physiological significance. Furthermore, they have a moderate polarity and therefore may be soluble in both aqueous and lipophilic media.

In accordance with yet another aspect of the invention there is provided a process for the synthesis of a compound of formula (I), comprising:

(a) protecting the 5′ and 6′ hydroxyl groups by reaction of ascorbic acid with a ketone of the general formula R⁷R⁸CO, wherein R⁷ and R⁸, the same or different, each is C₁-C₁₀ alkyl, to yield a compound of the general formula (II):

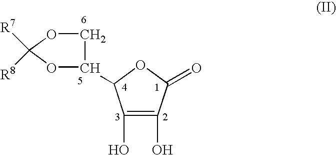

(b) reacting the resulting protected ascorbic acid molecule of formula (II) with a compound of the general formula R¹Hal, wherein R¹ is as defined before, and if it is an amino acid residue, the amino group thereof is protected, to yield a compound of the general formula (III):

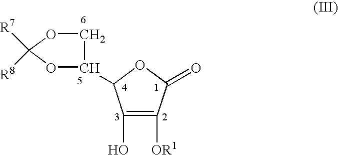

(c) reacting the compound of formula (III) with a concentrated carbonate solution of the metal R², to yield a compound of the general formula (IV):

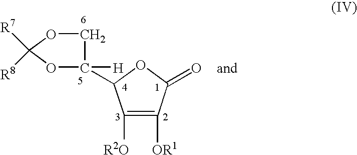

(d) hydrolyzing the compound of formula (IV) to deprotect the 5- and 6-hydroxyl groups, to yield a compound of the general formula (1) wherein R³ and R⁴ are both hydrogen.

The hydrolysis of the compound of formula (IV) is preferably carried out under mild conditions. If derivatization of positions 5 and/or 6 is desired, at the first step, the hydroxyl groups at positions 2 and 3 are protected, at the second step the hydroxyl groups at position 5 and/or 6 are etherified or esterified, and, at the third step, the hydroxyl groups at positions 2 and 3 are deprotected, followed by derivatization of the hydroxyl groups at positions 5 and/or 6 with a suitable reagent, e.g., an acyl chloride, to obtain a compound of the formula (I) wherein R³ and/or R⁴ are not hydrogen.

The novel compounds of formula (I) of the invention can be used for all uses known and to be discovered for stable ascorbic acid derivatives as a source of ascorbic for treatment of conditions, diseases and disorders in which ascorbic acid is therapeutically, nutritionally or cosmetically active. Thus, the novel derivatives of the invention are suitable as ingredients of compositions including cosmetic, cosmeticeutical, nutraceutical and pharmaceutical compositions, either as the sole active ingredient or in combination with other active ingredients.

In one embodiment, the invention provides pharmaceutical compositions comprising a compound of formula (1) together with a pharmaceutically acceptable carrier. These pharmaceutical compositions can be used for all conditions, diseases and disorders in which ascorbic acid is therapeutically active such as in the treatment of cancer, alone or together with other agents, e,g. lipoic acid (as described in U.S. Pat. No. 6,448,287) or a conventional chemotherapeutic agent, or for treatment of vitamin C deficiency in scurvy or for stimulation of the immune system.

In another embodiment, the invention provides cosmetic compositions comprising a compound of formula (I) together with a cosmetic applicable carrier, excipients or diluents. These compositions can be used for topical application for skin care, e.g. for reducing fine lines, wrinkles, skin roughness, for skin whitening, and stimulation of collagen synthesis as antiaging agent.

The cosmetic or dermatological compositions of the invention can be used for treatment of radiation-induced skin damage or photodamaged skin, particularly ultraviolet radiation-induced skin damage, e.g., sunburn, for skin damage resulting from chronic exposure to sunlight as well as in the natural aging process, where free radical-induced damage is involved and where collagen content is diminished.

Thus, the invention also relates to a method of skin care, particularly for reducing fine lines, wrinkles and skin roughness, for skin whitening or for stimulation of collagen synthesis in a mammal in need thereof, which comprises topically administering a composition comprising a compound of general formula (I) of the invention to said mammal in need.

In one preferred embodiment, the composition of the invention is a topical composition for skin application, both for pharmaceutical and cosmetic uses. These cosmetic and dermatological compositions may be used for an effective delivery of vitamin C to the skin. The cosmetic and dermatological compositions comprising a stabilized ascorbic acid derivative of formula (I) may be used as moisturizing, cream, anti-aging cream, anti-wrinkle cream, sunscreen cream, for stimulating collagen-production, and the like, alone or together with other active ingredients such as vitamin E, vitamin P, retinol, retinol esters, hyaluronic acid, and/or algal extracts.

The compositions may be prepared in various forms including, but not limited to lotions, creams, gels, ointments, salves, liquids, etc. The nature of the formulation may vary depending on the intended use in accordance with the overall polarity of the compound of formula (I). For example, a polar compound of formula (I) may be formulated in an aqueous formulation such as gel, while a more hydrophobic compound of formula (I) will be formulated in an emulsion form. The topical compositions will comprise carriers and excipients such as water, emulsifiers, natural oils, fats, antioxidants, stabilizers, moisturizers, gel-forming agents, preservatives and any other excipient suitable for the intended cosmetic or dermatological purpose as well known in the art.

The cosmetic compositions of the present invention may also include, for example, vehicles including, but not limited to, water or alcohol; humectants, including, but not limited to, glycerin; buffering agents including, but not limited to, citric acid and sodium citrate; viscosity adjusters, including, but not limited to, carbomer gelling agents, gum derivatives, and the like; preservatives including, but not limited to, methylparaben, propylparaben, and phenoxyethanol; emulsifiers including, but not limited to, polysorbitate 80 and glyceryl distearate; conditioning agents including, but not limited to, octyl hydroxystearate; emollients including, but not limited to, cholesterol NF, petrolatum, mineral oils and esters including, but not limited to, isopropyl myristate, isopropyl palmitate, 1-decene polymer (hydrogenated), and C₁₂-C₁₅ alkyl benzoates; antioxidants, including, but not limited to, antioxidant BHT, tocopheryl acetate, and the like; UV stabilizers; UV radiation absorbers (sunscreen filters); fragrances; colorants; or any combinations of any of the foregoing. In addition, pharmaceutical or nutraceutical compositions in accordance with the invention may also be formulated for oral administration with a pharmaceutically active carrier for use in all cases where there is a requirement for supplemental administration of vitamin C, both in cases of vitamin C deficiency or when increased amounts are necessary to strengthen the immune system or any other condition in which vitamin C may be beneficial.

The nutraceutical compositions according to the invention may optionally comprise other nutraceutics such as, but not limited to, vitamins, amino acids, hormones, prohormones, and minerals.

For oral administration, the compounds of the invention may be formulated in capsules, tablets or liquids. In tablets, for example, the active ingredient is mixed with a carrier having the necessary compression properties in suitable proportions and compacted in the shape and size desired. Solid carriers include, for example, calcium phosphate, magnesium stearate, talc, sugars, lactose, dextrin, starch, gelatin, cellulose, methylcellulose, sodium carboxymethylcellulose and poly(vinyl pyrrolidone). Liquid formulations may be used by preparing solutions, suspensions, emulsions or syrups. The active ingredient can be dissolved or suspended in a pharmaceutically acceptable liquid solvent such as water, an organic solvent, and a mixture of pharmaceutically acceptable oils or fats. Suitable liquid carriers for oral administration include water, alcohols, and oils. The composition may be in the form of microcapsules or nanocapsules confining the compound of the invention.

The invention will now be illustrated by the following non-limiting Examples.

### EXAMPLES

### Example 1

### Synthesis of Sodium Salt of 2-Capryloyl Ascorbic Acid

Step 1. Synthesis of 5,6-isopropylidenyl ascorbic acid.

20 g (0.125 mol) of anhydrous cupric sulfate were added to a suspension of 20 g (0.114 mol) of ascorbic acid in 660 mL of dry acetone. The reaction mixture was stirred for 20 h at room temperature. The process was monitored by TLC (chloroform-methanol-water, 10:10:3). After filtration and evaporation 22.57 g (92%) of 5,6-isopropylidenyl ascorbic acid were obtained.

Step 2. Synthesis of 2-capryloyl-5,6-isopropylidenyl ascorbic acid.

Capryloyl chloride (12.0 g, 0.074 mol) was added dropwise at 0° C. to a solution of 5,6-isopropylidenyl ascorbic acid (14.5 g, 0.067 mol) in dry pyridine (80 mL). The reaction system was stirred for 1.5 h at 0° C., and the process was monitored by TLC (chloroform-methanol, 3:1). Afterwards, ice water (300 mL) was added and the reaction mixture was adjusted to pH 3 using phosphoric acid (˜10 mL) and extracted with ethyl acetate (2×100 mL). Combined extracts were washed with a saturated solution of sodium chloride up to pH 7. The washed organic layer was dried with anhydrous MgSO₄ and concentrated by vacuum. The residue was washed with hexane, and concentrated by vacuum to give 22.9 g (89%) of 2-capryloyl-5,6-isopropylidenyl ascorbic acid.

Step 3. Synthesis of sodium salt of 2-capryloyl-5,6-isopropylidenyl ascorbic acid.

3.0 g of 2-capryloyl-5,6-isopropylidenyl ascorbic acid were dissolved in 150 mL of ethyl acetate and put into the separated funnel. 50 mL of 3 M solution of sodium carbonate were added to the prepared solution. After mixing and exposing for about 10 min, a triple-phase system was obtained. The intermediate phase was selected, filtered and concentrated. The yield of the product equals 65-70%. The sodium salt of 2-capryloyl-5,6-isopropylidenyl ascorbic acid was obtained as light-yellow solid amorphous substance.

Step 4. Synthesis of sodium salt of 2-capryloyl ascorbic acid

The end product is obtained by deprotection of the 5- and 6-hydroxyl groups of the compound of Step 3, at mild conditions, by means of the reaction mixture MeOH:H₂O:2N HCl=30:2:1 (v/v/v) at 4° C. for 24 h followed by washing with sodium chloride up to pH 7, and thorough vacuum drying of the product in order to remove organic solvent. Yield: about 90%.

### Example 2

### Synthesis of Sodium Salt of 2-Palmitoyl Ascorbic Acid

Step 1. Synthesis of 5,6-isopropylidenyl ascorbic acid.

20 g (0.125 mol) of anhydrous cupric sulfate were added to a suspension of 20 g (0.114 mol) of ascorbic acid in 660 mL of dry acetone. The reaction mixture was stirred for 20 h at room temperature. The process was monitored by TLC (chloroform-methanol-water, 10:10:3). After filtration and evaporation 22.57 g (92%) of 5,6-isopropylidenyl ascorbic acid were obtained.

Step 2. Synthesis of 2-palmitoyl-5,6-isopropylidenyl ascorbic acid.

Palmitoyl chloride (12.0 g, 0.074 mol) was added dropwise at 0° C. to a solution of 5,6-isopropylidenyl ascorbic acid (14.5 g, 0.067 mol) in dry pyridine (80 mL). The reaction system was stirred for 1.5 h at 0° C., and the process was monitored by TLC (chloroform-methanol, 3:1). Afterwards, ice water (300 mL) was added and the reaction mixture was adjusted to pH 3 using phosphoric acid (˜10 mL) and extracted with ethyl acetate (2×100 mL). Combined extracts were washed with saturated solution of sodium chloride up to pH 7. The washed organic layer was dried with anhydrous MgSO4 and concentrated by vacuum. The residue was washed with hexane, concentrated by vacuum to give 22.9 g (89%) of 2-palmitoyl-5,6-isopropylidene ascorbic acid.

Step 3. Synthesis of sodium salt of 2-palmitoyl-5,6-isopropylidenyl ascorbic acid.

3.0 g of 2-palmitoyl-5,6-isopropylidenyl ascorbic acid were dissolved in 150 mL of ethyl acetate and put into the separated funnel. 50 mL of 3 M solution of sodium carbonate were added to the prepared solution. After mixing and exposing for about 10 min the triple-phase system was obtained. The intermediate phase was selected, filtered and concentrated. The yield of the final product equals 65-70%. The sodium salt of 2-palmitoyl-5,6-isopropylidenyl ascorbic acid was obtained as light-yellow solid amorphous substance.

Step 4. Synthesis of sodium salt of 2-palmitoyl ascorbic acid.

The end product is obtained by deprotection of the 5- and 6-hydroxyl groups of the compound of Step 3, at mild conditions, by means of the reaction mixture MeOH:H₂O:2N HCl=30:2:1 (v/v/v) at 4° C. for 24 h followed by washing with sodium chloride up to pH 7, and thorough vacuum drying of the product in order to remove organic solvent. Yield: about 90%.

In the same way as described in Examples 1-2, other compounds are obtained when the metal is K, Ca or Mg, for example, and the esters at position 2 are from other carboxylic acids, preferably fatty acids, or from amino acids such as glycine or alanine.

### Example 3

### Dermatological Effect—Stimulation of Collagen Synthesis in Primary Human Foreskin Fibroblasts by Ascorbic Acid Derivatives

L-Ascorbic acid stimulates collagen synthesis in cultured human skin fibroblasts. Ascorbate contributes to several metabolic processes including efficient hydroxylation of hydroxyproline in collagen synthesis.

In order to evaluate the effect of the ascorbic acid derivatives of the invention on collagen synthesis, cultured human foreskin fibroblasts are placed in 24-well microculture plates in DMEM supplemented with 10% fetal calf serum containing 100 μg/ml beta-aminopropionitrile, 10 μCi [2,3-³H]proline, in the presence of either ascorbic acid (positive control) or the ascorbic acid derivative of Example 1 or 2 in different concentrations, e.g. from 1 mM to 50 mM. The cultures are incubated for 24 hours. The [2,3-³H]-proline incorporation into pepsine-resistant salt precipitated extracellular collagen is determined and used as an index of efficiency of the collagen synthesis. The results are averaged from four identically treated wells and corrected for cell number in sample. The ascorbic acid derivatives are expected to show an effectiveness comparable to that of L-ascorbic acid or better, on collagen synthesis.

1. A compound of the general formula I: 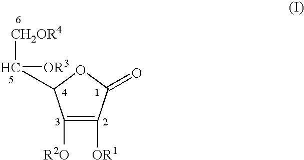
wherein R¹ is a C₂-C₂₂ acyl group, an amino acid group, or a C₁-C₁₇ alkyl group; R² is ammonium or a metal cation; and each of R³ or R⁴, independently, is hydrogen, a C₂-C₂₂ acyl group, an amino acid residue, or a C₁-C₁₇ alkyl group.

2. A compound according to claim 1 wherein R² is ammonium or a monovalent metal cation selected from Na⁺, K⁺, or Li⁺.

3. A compound according to claim 1 wherein R² is a divalent alkaline earth metal cation selected from Mg⁺⁺, Ca⁺⁺, or Ba⁺⁺ or a trivalent metal cation selected from Al⁺⁺⁺ or Fe⁺⁺⁺.

4. A compound according to claim 1 wherein R² is selected from Na⁺, K⁺, Mg⁺⁺, or Ca⁺⁺.

5. A compound according to claim 1 wherein R¹ is a C₂-C₂₂ carboxylic acyl group derived from a straight or branched, saturated or unsaturated carboxylic acid selected from acetic, propionic, n-butyric, isobutyric, sorbic, n-valeric, isovaleric, caproic, caprylic, capric, undecanoic, lauric, tridecanoic, myristic, pentadecanoic, or arachidonic acid.

6. A compound according to claim 5 wherein R¹ is a C₈-C₁₈ acyl group.

7. A compound according to claim 6 wherein R¹ is capryloyl or palmitoyl.

8. A compound according to claim 1 wherein R¹ is the residue of a natural α-amino acid such as glycine, alanine, serine, leucine, isoleucine, phenylalanine, tyrosine or glutamic acid.

9. A compound according to claim 1 wherein R¹ is the residue of glycine.

10. A compound according to claim 1 wherein R³ and R⁴ are hydrogen, R² is ammonium or a metal cation, and R¹ is a C₈-C₁₈ acyl derived from a fatty acid.

11. A compound according to claim 10 wherein R² is a monovalent metal cation selected from Na⁺, K⁺, or Li⁺ or a divalent metal cation selected from Mg⁺⁺ or Ca⁺⁺.

12. The compound according to claim 1 wherein R³ and R⁴ are hydrogen, R² is Na⁺ and R¹ is capryloyl.

13. The compound according to claim 1 wherein R³ and R⁴ are hydrogen, R² is Na⁺ and R¹ is palmitoyl.

14. A composition comprising as an active ingredient a compound according to claim 1.

15. A pharmaceutical composition according to claim 14 for treatment of conditions, diseases or disorders associated with vitamin C deficiency and/or where a supplemental administration of vitamin C may be beneficial.

16. A pharmaceutical composition according to claim 14 for treatment of cancer.

17. A pharmaceutical composition according to claim 16 further comprising lipoic acid or a chemotherapeutic agent.

18. A pharmaceutical composition according to claim 15 for enhancement of the immune system.

19. A nutraceutical composition according to claim 14, optionally comprising other nutraceutics.

20. A pharmaceutical or nutraceutical composition according to claim 14 adapted for oral administration, optionally in the form of micro/nanocapsules confining an effective amount of the compound of formula (I).

21. A pharmaceutical composition according to claim 14 for topical use.

22. A cosmetic composition according to claim 14 together with cosmetic applicable carriers, excipients or diluents.

23. A cosmetic composition according to claim 22 for topical application for skin care.

24. A cosmetic composition according to claim 23 for reducing fine lines, wrinkles or skin roughness, skin whitening or stimulation of collagen synthesis.

25. A cosmetic composition according to claim 24 for use as anti-aging agent.

26. A cosmetic composition according to claim 24 for treatment of photodamaged skin.

27. A cosmetic composition according to claim 22 in the form of a lotion, cream, gel, or in liquid form.

28. The cosmetic composition according to claim 22 for use as moisturizing cream, anti-ageing cream, anti-wrinkle cream, sunscreen cream, skin whitening and for stimulating collagen synthesis.

29. The cosmetic composition according to claim 22 further comprising one or more active ingredients such as vitamin E, vitamin P, retinol, retinol esters, hyaluronic acid, and/or algal extracts.

30. A dermatological composition according to claim 14 for treating or controlling skin states or conditions associated with vitamin C deficiency and/or where a supplemental administration of vitamin C may be beneficial.

31. A method of skin care, particularly for reducing fine lines, wrinkles and skin roughness, or for stimulation of collagen synthesis in a mammal in need thereof, which comprises topically administering a composition comprising a compound of general formula (I) of claim 1 to said mammal in need.
